(19) United States
(12) Patent Application Publication
(10) Pub. No.: US 2004/0152685 A1
(43) Pub. Date: Aug. 5, 2004

(54) Agents and methods for the prevention of initial onset and recurrence of existing cancers

(76) Inventor: Tom Slaga, Holden, CO (US)

Correspondence Address: Davis G Henry, 900 Washington Avenue, PO Box 1470, Waco, TX, 76703-1470, US

(21) Appl. No.: 10/471,781

(22) PCT Filed: Mar. 13, 2002
(86) PCT No.: PCT/US02/07445

(30) Foreign Application Priority Data
Mar. 14, 2001 (US) 09808408

(51) Int. Cl. A61K 31/56

(52) U.S. Cl. 514/182

(57) ABSTRACT

The use of 2-methoxyestradiol, analogues of 2-methosyestradiol, their method of synthesis and therapeutic use, and the use of combinations of the 2 methoxyestradiol and its analogues with synergistic compounds (namely eugenol), all in the prevention of initial onset cancers and the recurrence of previously existing cancers.

### CITATION TO PRIOR APPLICATION

This is a continuation-in-part with respect to U.S. application Ser. No. 09/527,283, filed Mar. 17, 2000 from which priority is claimed under 35 U.S.C. §120, and a continuation application with respect to U.S. application Ser. No. 09/808,408, filed Mar. 14, 2000, from which priority is claimed under provisions of the Patent Cooperation Treaty.

### BACKGROUND OF THE INVENTION

A. Field of the Invention

The present invention concerns the novel use of known chemical compounds, the chemical synthesis and use of novel chemical compounds, and the use of novel combinations of both new and existing compounds, all in the prevention of initial onset and recurrence of a broad array of cancers.

B. Background of the Invention

1. The Problem: Primary Modalities of Cancer Cell Growth and Expansion (New, Existing and Recurring Cancers)

Cancer is the second leading cause of death in the United States, accounting for approximately one in four deaths. Recent estimates by the American Cancer Society suggest that in excess of 500,000 people die from cancer every year—that is approximately 1,500 deaths a day. Further, approximately 2.5 million new cases of cancer were expected to be diagnosed in the year 2000 alone. At an estimated annual cost of $107 billion dollars in health care costs and lost productivity due to death and illness, cancer inflicts a vast human and monetary toll on the United States.

The generic use of the term “cancer” only hints at the vast diversity of anatomical structures that this disease affects and the myriad of molecular bases that form the foundation of this disease. The collective use of the word cancer includes diseases affecting the brain, breast, cervix uteri, colon, corpus uteri, kidney, renal pelvis, larynx, lung, bone marrow, bronchus, skin, lymph system, nervous system, oral cavity, pharynx, ovary, pancreas, prostate, rectum, stomach, testis, thyroid, urinary bladder, and others. The individual molecular bases of these diverse afflictions can be varied and diverse. However, among this diverse field of afflictions, there exist two unified modalities of cell growth and/or proliferation that are common to almost all types of cancer: 1) unchecked cell growth and/or immortality, and 2) angiogenesis.

On of the problems that characterize a vast number of cancers is the unregulated growth or unchecked life span of aberrant cells in the various tissues of the body. Normal cells grow, divide, and die on a regular basis. The process by which cells normally die is called apoptosis. However, when normal cell growth and death become unchecked in the body, by any number of processes, such unchecked growth and/or immortality leads to destroy the regular functioning of the various tissues of the body. Such growth or immortality can ultimately lead to the occurrence of a host of solid tumors, leukemia's, lymphomas, or the metastasis of cancer cells throughout the body. Unchecked cell growth and/or immortality are problematic biological mechanisms common to almost all types of cancer.

Another biological mechanism that is common to, and problematic in the prevention or treatment of, all solid cancer tumors is angiogenesis. Angiogenesis refers to the process by which new blood vessels are formed in the body. Without a dedicated blood supply, solid tumors have only limited growth potential—perhaps 2 mm in diameter maximum. However, angiogenesis often occurs in cancerous tissues and tumors, thus enabling solid tumors to sequester greater amounts of nutrients from the body and allowing them to proliferate rapidly, even spreading to other parts of the body. Angiogenesis is a critical means by which solid tumors grow rapidly and metastasize, hastening the process of death or disfigurement.

These two independent biological mechanisms are the common, primary modalities by which almost all cancer cells proliferate and grow. Hence, a novel approach for the treatment of cancer would be the development of pharmacological agents that have dual roles as anti-angiogenic as well as pro-apoptotic agents. Such an agent will have the ability to target both components of a cancer: kill the tumor cell by induction of apoptosis and cut off the blood supply to the tumor cell so that it will not grow.

In addition to the dire need for effective treatment modalities for existing cancers, there is arguably an even greater need for effective cancer preventative means, both with respect to initial onset of cancers, as well as in the context of recurrence of cancers after operative intervention.

A recent breakthrough in the treatment of cancer is the use of 2-methoxyoestradiol (hereinafter “2-ME”). 2-ME is an endogenous non-toxic metabolic byproduct of estrogens that is present in human urine and blood. (1) A potential role for 2-ME as a chemopreventive agent has been reported in the mammary and pancreatic models. (2) 2-ME has also been shown to inhibit endothelial cell proliferation implicating its potential role in angiogenesis. (3) In addition, apoptosis has been implicated as a mechanism for 2-ME's cytostatic and anti-angiogenic effect.

### SUMMARY OF THE INVENTION

It is an object of the present inventionto provide an agent or composition, or more than one agent or composition, that is efficacious in inhibiting the proliferation and/or angiogenesis of cancer cells.

It is another object of the present invention to provide a method for creating novel molecules that are efficacious in inhibiting the proliferation and/or angiogenesis of cancer cells.

It is another object of the present invention to provide a composition the primary active ingredient of which are an analogue or analogues of 2-methoxyestradiol which are efficacious in inhibiting the proliferation and/or angiogenesis of cancer cells.

It is another object of the present invention to provide a method for inhibiting the proliferation and/or angiogenesis of cancer cells through use of a composition the primary active ingredient of which is 2-methoxyestradiol or an analogue thereof, as described herein.

It is, therefore, an object of the present invention to provide a new modality for the prevention of cancers as well as the suppression of recurrence of cancers once treated.

It is another object of the present invention to provide a new modality for the prevention of cancer as well as the suppression of recurrence of cancers once treated.

It is another object of the present invention to provide a new modality for the prevention of prostate cancer as well as the suppression of recurrence of prostate cancer once treated.

It is another object of the present invention to provide a method by which the known substance of 2-ME may be employed in a new and unobvious manner in the prevention of initial onset and of recurrence of cancers, including prostate cancer.

It is another object of the present invention to provide a method by which the known substance of 2-ME, alone, or in combination with synergistic compounds, including eugenol and certain other herein disclosed compounds, may be employed in the prevention of initial onset and post-operative recurrence of cancers, including prostate cancer.

In satisfaction of these and related objects, disclosed and claimed herein is the use of 2-ME or 2ME analogues, alone, or in combination with synergistic compounds, including eugenol and certain other herein disclosed compounds, in the prevention of initial onset and post-operative recurrence of cancers, including prostate cancer. Also, because of the mode of action (anti-tumorigenic, anti-angiogenic, and pro-apoptotic), 2-ME or certain herein disclosed derivatives, with or without the herein described synergistic compounds, can be used, not only in the treatment of any kind of tumor, including prostate, brain, liver, lung, colon, and skin, but in preventing initial onset, or recurrence of such cancers after treatment.

Findings by the present inventors pertaining to the mechanisms of action of 2-ME, its derivatives, and the synergistic compounds herein described indicate that these compounds serve as cancer preventative agents, as well as curative agents. The present inventors previous work, filed with the original patent application and another continuation-in-part application, shows that 2-ME is of great significance in the treatment of prostate, brain, and nervous system cancer through the induction of apoptosis. This body of work indicates that 2-ME is an anti-tumorigenic agent with a significant therapeutic advantage since it can preferentially inhibit actively proliferating cells (characteristic of tumor cells) without affecting the growth of normal cycling cells. Additionally, 2-ME appears to also inhibit the formation of new blood vessels. To the best of our knowledge, this is the first compound that targets two components of cancer: the tumor cells and their blood supply. The present inventors have demonstrated that 2-ME is a chemical compound with a significant role as an antitumorigenic agent with broad efficacy in a variety of cancerous cell populations.

Building on these findings, further experiments have helped to elucidate the structural bases for 2-ME's molecular efficacy. A number of experiments have been conducted using 2-ME and 16-epiestriol (hereinafter “16-ES”), an analogue of 2-ME that lacks the methoxy group at the second position. In a multitude of experiments, using prostate cancer cell lines (both androgen-dependent (LNCaP), and androgen-independent (DU145) cells), and a brain and/or nervous system cancer cell line (DAOY), the present inventors have studied the effects of 2-ME and 16-ES on cell proliferation and the induction of apoptosis, in a number of ways. The sum of all the data clearly indicates that 2-ME is a compound that significantly inhibits cancerous cell growth and has pro-apoptotic effects, while 16-ES does not. In total, these data show that the efficacy of 2-ME is associated with the methoxy moiety at the second position of 17β-estradiol (E₂). Further, it also indicates efficacy of a series of compounds with various moieties at the second position in the treatment of cancer. Additionally, the specific anti-proliferative, pro-apoptotic, anti-angiogenesis, and other efficacy of 2-ME against cancer cells indicates that other structural modifications of the molecule will reasonably be expected to increase the efficacy of the agent. Thus, the present inventors now propose a method of synthesizing a number of analogues of 2-ME that will exceed the efficacy of 2-ME in the prevention of cancer.

Further still, the investigations of the present inventors indicate that 2-ME (and predictably its analogs) work synergistically with other compounds, notably eugenol, to achieve even greater results in the same manner and modality as 2-ME alone in attacking cancer cells and preventing initial cancer formation and recurrence of cancer.

### DETAILED DESCRIPTION OF THE PREFERRED EMBODIMENT

Data from the present inventors laboratory shows that 2-ME inhibits the growth of brain, nervous system and prostate cancer cells but that 16-epiestriol does not. This indicates that substituting the second position of 17b-estradiol (E₂) with a methoxy group generates a molecular structure that shows significant and selective growth inhibitory activity toward prostate cancer cells while simultaneously eliminating the potentially detrimental growth stimulating activity of E₂ itself. The analogues of 2-ME to be prepared as described below are designed (1) to determine which components of the 2-ME molecule in addition to the 2-methoxy group are required for the observed chemopreventive effects and (2) to determine if growth-inhibitory 2-ME analogues can be created that are effective.

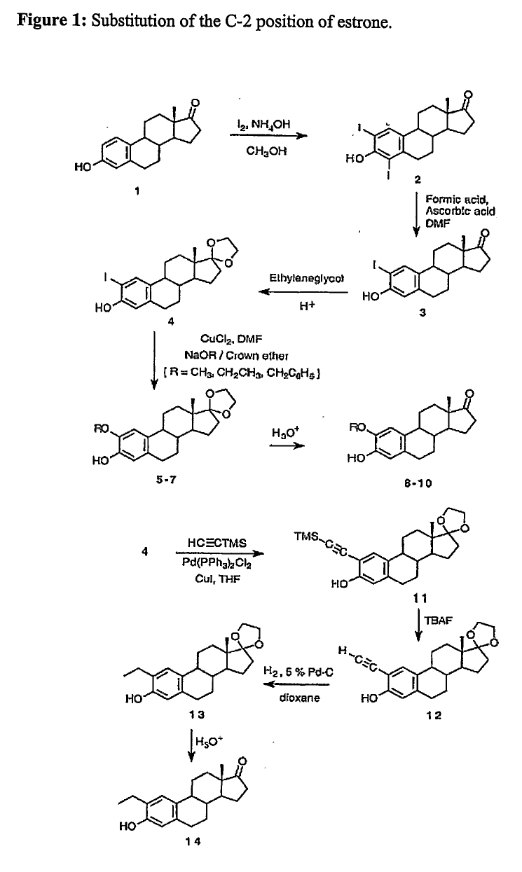

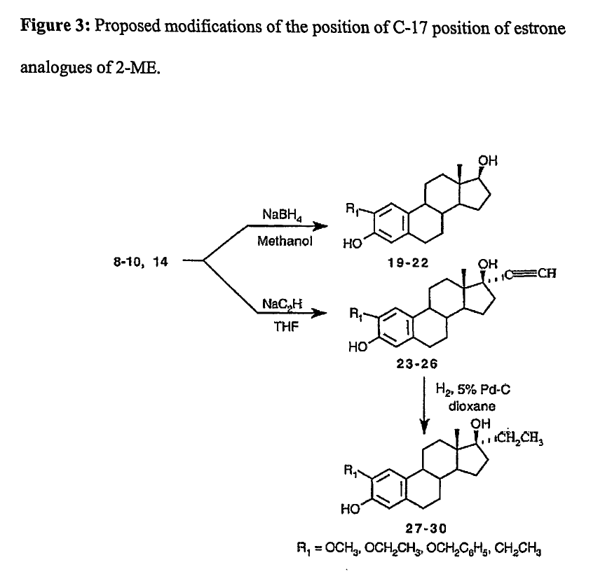

The initial compounds to be synthesized will be 2 alkoxy substituted analogues of estrone shown in FIG. 1. These compounds will then be converted into the 2-ME analogues as shown in FIG. 3 (analogues 19-21, 23-25, and 27-29).

FIG. 1 illustrates how the A ring of the E₂ steroidal nucleus will be modified to generate 2-alkoxy substituted analogues of estrone (analogues 8-10) and a 2-ethyl substituted estrone analogue (analogue 14). The key reactions in this figure are the synthesis of compound 2, 2,4-diiodoestrone, and its conversion to compound 3, the 2-iodoestrone derivative. The iodination and diodination of the estrone starting material (analogue 1) will be carried out as described by Ikegawa et al in their synthesis of catecholic equilin and equilin derivatives. (4) The proposed conversion of the ethylenedioxy protected 2-iodoestrone derivative 4 to the protected 2-methoxy, 2-ethoxy, and 2 benzyloxy derivatives 5-7 by Cu (I) catalyzed reactions of the alkoxides in dimethyformamide in the presence of a crown ether is based upon the comparable reaction of the protected 2-iodoequilin also described by Ikegawa et. al in the synthesis of catechol equilins. (4) It should be noted that if it proves necessary the estrone starting material used in FIG. 1 could be protected as the ethylenedioxy derivative by treatment with ethylene gylcol prior to the iodination reaction. The Pd(Ph₃)Cl₂/CuI catalyzed coupling of the aryl iodide (analogue 4) with trimethylsilyl substituted acetylene to yield the 2-alkynyl substituted estrone derivative 11 shown in FIG. 1 has many known procedents (5). The present inventors have carried out many such coupling reactions in their laboratory and have found that molecules containing active hydrogens (NH₂ or OH groups) can be successfully coupled in such reactions if care is taken to form the reactive Cu-TMS acetylene complex before the halogenated aromatic substrate is added. It is therefore anticipated that this reaction will proceed as shown in FIG. 1. If, however, the reaction fails to be successful as shown in FIG. 1, the intermediate 4 will be coupled with trimethylsilylacetylene in 9:1 CH₃CN/H₂0 catalyzed with Pd(AcO)₂/PPh₃/CuI. The present inventors have carried out a model reaction in their laboratory with an unprotected iodophenol that gave the desired coupling product with this procedure.

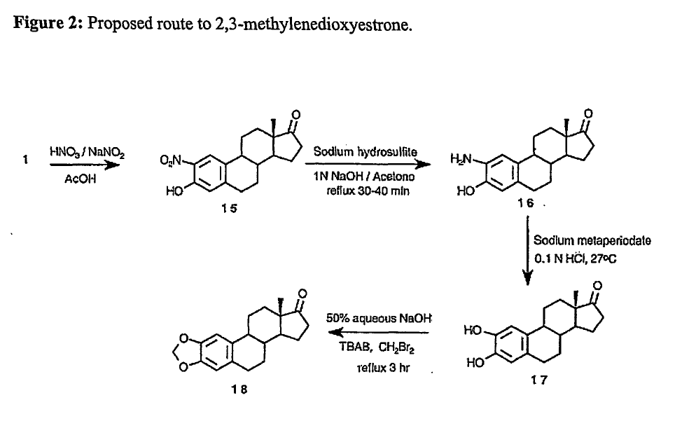

FIG. 2 outlines the reaction sequence that will be employed to prepare that 2,3-methylenedioxyestrone derivative (analogue 18). The reaction sequence is based upon the reaction sequence employed by Stubenrauch and Knuppen to prepare catechol estrogens. (6)

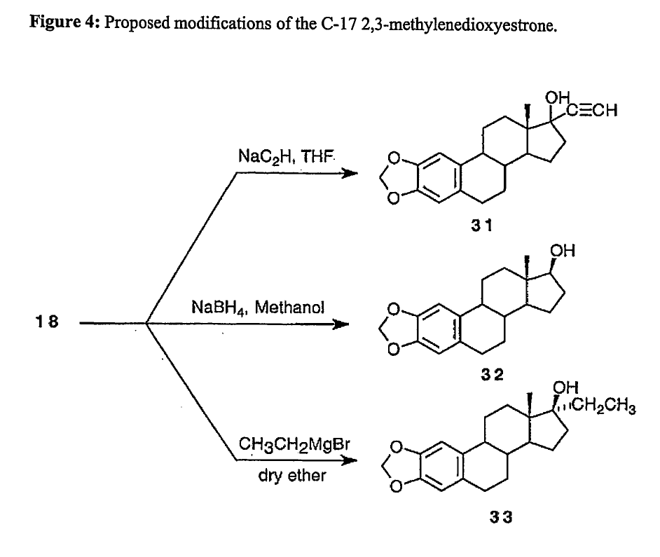

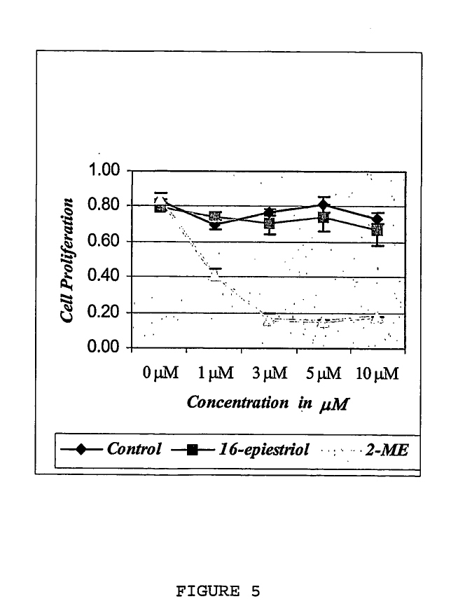

FIGS. 3 and 4 illustrate how 2-methoxyestrone and the 2methoxyestrone analogues prepared as outlined in FIGS. 1 and 2 above will be converted into (i) 2-methoxyestrone methoxyestrone and its analogues and (ii) 2,3-methylenedioxyestrone analogues modified at position C-17. The preparation of these structures will not only allow us to test the requirement for the 17b-hydroxyl group in the chemopreventive activity of 2-ME but will also enable us to determine if substitutions at C-17 (for example, the 17-ethynyl-2-ME derivative, 23) will decrease the rate of metabolism and deactivation of 2-ME and its analogues. As outlined in FIGS. 3 and 4 below, the present inventors propose to prepare both 2-ethyl-17b-estradiol (analogue 22) and 2,3-methylenedioxy-17b-estradiol (analogue 32). In addition, since 17a-ethynylestradiol (ethynylestradiol) is both a potent estrogenic and long-lived analogue of E₂, the 17a-ethynyl derivative of 2-ME (analogue 19) will be prepared as outlined in FIG. 3. In addition, by directing synthesis to produce estrone analogues of the target structures (analogues 8-10, 14, and 18) as illustrated in FIGS. 1 and 2, it will be possible to prepare 17a-ethynyl, and 17a-ethyl derivatives of the 2-alkoxy, 2-ethyl, and 2,3-methylenedioxy analogues (analogues 23-26, 27-30, 31 and 32).

It should be noted that the proposed reactions used to modify the C-17 carbonyl of the estrone analogues shown in FIGS. 3 and 4 are standard reactions that have been successfully applied to estrone. (7)

Although not explicitly shown in FIGS. 1 and 3, the 2-ethynyl intermediate shown in FIG. 1 (analogue 12) will also be converted into 2-ethynylestrone and 2-ethynylestradiol for testing. Further, although not explicitly indicated in FIGS. 1 and 2, the 2-ethynylestrone derivative 11 shown in FIG. 1 will also be converted into 2-ethynylestrone and 2-ethynylestradiol as shown in FIG. 2 for the other intermediates. This will generate two additional 2-ME analogues for biological testing. Lastly, it is also possible to modify the acetylene coupling reaction shown in FIG. 1 to prepare 2-(1-propynyl) and 2-(1-butynyl) derivatives of 2-ME that could serve as precursors of 2-propyl and 2-butyl 2-ME analogues.

The synthesis reactions in FIGS. 1-4 outlined above will provide an efficient way of generating 2-ME (analogue 19) and fourteen 2-ME analogues (analogues 20-33) that can be utilized to determine the effects of modifying both the C-17 and the C-2 position of 2-ME. Samples of the estrone analogues themselves (analogues 8-10, 14, 18) will also be tested for their potential growth-inhibitory activity. The reaction sequences outlined in FIGS. 1-4 will therefore produce a total of 21 new 2-ME analogues to be tested as potential selective inhibitors of cancer cell growth and angiogenesis. It is anticipated that one or more of these analogues may manifest selective growth-inhibitory activities towards cancer cells while, at the same time, being less subject to metabolic conversions that will deactivate or eliminate these active analogues. It is also likely that 17a-ethynyl derivative of 2-ME may have a longer effective half-life both in vitro and in vivo.

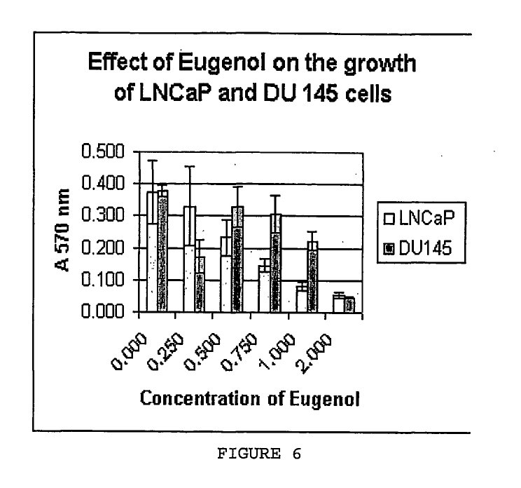

Referring to FIG. 6, eugenol also inhibits the growth of LNCaP cells significantly. A concentration of approximately 0.75 mM was necessary to see 50% inhibition of growth of LNCaP cells whereas a concentration of more than 2 mM was necessary to see similar effect in DU145 cells.

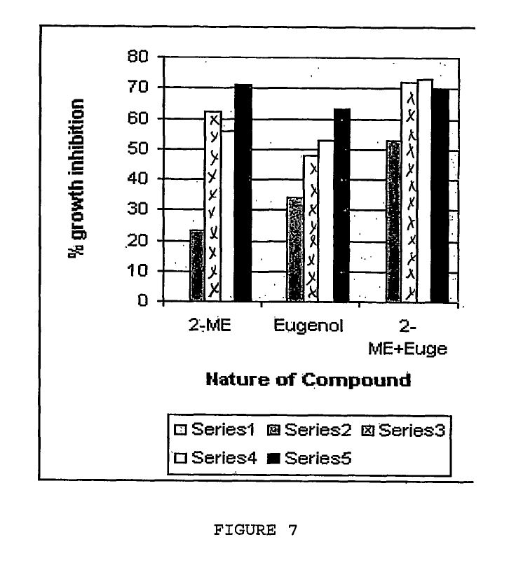

The investigational work of the present inventors establish that eugenol works in combination with 2-ME to achieve even more impressive results than either substance alone. Cells were treated with either eugenol (0.25, 0.5, 0.75 or 1 mM) or 2-ME (0.5, 1, 2 or 3 mM) or both (0.25, 0.5, 0.75 or 1 mM of eugenol along with 0.5 mM of 2-ME). Cell growth was measured following 72 hours of treatment as described above. As shown in FIG. 7, 0.5 mM of 2-ME inhibited growth of LNCaP cells by about 20% and 0.25 mM of eugenol inhibited the growth by about 30%. However, combining both the agents showed more than 50% inhibition thereby establishing a synergistic activity of eugenol and 2-ME in combating cancer cells.

The mechanisms of action at work against the cell lines investigated thus far are reasonably expected to be equally efficacious in treating other cancers and pre-cancerous conditions, such BPH and the cancers of brain, liver, lung, colon and skin, and in preventing initial onset of cancers and preventing recurrence of cancers after treatment (such as prostectomies). Since both hormone-responsive and hormone-refractory prostate cancer cells are inhibited by 2-ME and its analogs, with or without synergistic compounds such as eugenol, patients can be treated with these agents after surgery to prevent the recurrence of hormone-refractory cancer.

Additionally, the analogues of 2-ME described above are expected to provide even greater efficacy, along and in combination with synergistic, similarly structured compounds as eugenol. This expectation is well-founded on the efficacy indications established for 2-ME and the effect of the above-taught structural changes to 2-ME as indicated by the work of the present inventors.

Application to existing, in vivo tumors may be of varying means, including, but not limited to, direct injection of the herein described agents, electrophoresis, and non-electromotive transdermal migration. Practitioners skilled in the use of chemopreventative agents will adjust dosages to meet the apparent needs of any particular patient, and the disclosure contained herein shall provide an enabling disclosure for the use of 2-ME and its analogs respectively alone, and with the synergistic compound of eugenol in the prevention of cancerous tumors as well as the suppression of recurrent cancers after treatment such as surgery.

References:

1. Gelbke, H. P., and Knuppen, R. 1976. The exertion of five different 2-hydroxyestrogen monomethyl ethers in human pregnancy urine. J Steroid Biochem. 7: 457-463.

2. Zhu, B. T. and Conney, A. H. 1998. Is 2-methoxyestradiol an endogenous estrogen metabolite that inhibits mammary carcinogenesis. Cancer Res. 58: 2269-2277.

3. Fotsis, T., Zhang, Y., Pepper, M. S., Adlercreutz, H., Montesano, R., Nawroth, P. P. and Schweigerer, O L. 1994. The endogenous estrogen metabolite 2-methoxyestradiol inhibits angiogenesis and suppresses tumor growth. Nature. 368: 237-239.

4. Ikegawa, S., Kurosawa, T., and Tohma, M. (1988) Syntheses of C-2caecholic equilin and equilin derivatives for use in metabolic studies. Chem. Pharm Bull. 36:2993-2999.

5. Neenan, T. X., and Whitesides, G. M. (1988) Synthesis of high carbon monomers bearing multiple ethynyl groups. J. Org. Chem., 53:2489-2496.

6. Stubenrauch, G. And Knuppen, R. (1976) Convenient large scale preparation of catechol estrogens. Steroids, 28:733-741.

7. Fieser, L. F. And Fieser, M. (1959) Estrogens in Steroids, Chapter 15, 444-502, Chapman and Hall, Ltd. London.

We claim:

1. The use of compositions useful in the inhibition of cancerous cell proliferation selected from a group consisting of:
the 2-ethyl-17-β-estradiol molecules identified as analogues 20-22 in FIG. 3, specifically excluding any claim to 2-methyloxyestradiol;
the 17-α-ethynyl molecules identified as analogues 23-26 in FIG. 3;
the 17-α-ethyl molecules identified as analogues 27-30 in FIG. 3;
the 2,3-methylenedioxy molecules identified as analogues 31, 32, and 33 in FIG. 4;
the 2-alkoxy substituted analogues of estrone molecules identified as analogues 8-10 in FIG. 1;
the 2-ethyl substituted molecule identified as analogue 14 in FIG. 1; and
the 2,3-methylenedioxyestrone molecule identified as analogue 18 in FIG. 2.

2. A method for inhibiting cancerous cell proliferation comprising the steps of:
selecting a composition from the group consisting of the 2-ethyl-17-β-estradiol molecules identified as analogues 20-22 in FIG. 3, specifically excluding any claim to 2-methyloxyestradiol, the 17-α-ethynyl molecules identified as analogues 23-26 in FIG. 3, the 17-α-ethyl molecules identified as analogues 27-30 in FIG. 3, the 2,3-methylenedioxy molecules identified as analogues 31, 32, and 33 in FIG. 4, the 2-alkoxy substituted analogues of estrone molecules identified as analogues 8-10 in FIG. 1, the 2-ethyl substituted molecule identified as analogue 14 in FIG. 1, or the 2,3-methylenedixoyestrone molecule identified as analogue 18 in FIG. 2; and
administering said composition to cells in which is identified suspected cancer cells.

3. The method of claim 2 wherein said suspected cancer cells are brain cancer cells.

4. The method of claim 2 wherein said suspected cancer cells are nervous system cancer cells.

5. The method of claim 2 wherein said suspected cancer cells are brain cancer cells and nervous system cancer cells.

6. The method of claim 2 wherein said suspected cancer cells are prostate cancer cells.

7. A composition for application to cancerous cells consisting in active constituents substantially of one or more agents chosen from the 2-ethyl-17-β-estradiol molecules identified as analogues 20-22 in FIG. 3, specifically excluding any claim to 2-methyloxyestradiol, the 17-α-ethynyl molecules identified as analogues 23-26 in FIG. 3, the 17-α-ethyl molecules identified as analogues 27-30 in FIG. 3, the 2,3-methylenedioxy molecules identified as analogues 31, 32, and 33 in FIG. 4, the 2-alkoxy alkoxy substituted analogues of estrone molecules identified as analogues 8-10 in FIG. 1, the 2-ethyl substituted molecule identified as analogue 14 in FIG. 1, or the 2,3-methylenedioxyestrone molecule identified as analogue 18 in FIG. 2.

8. The method of claim 8 wherein said suspected cancer cells are brain cancer cells.

10. The method of claim 8 wherein said suspected cancer cells are nervous system cancer cells.

11. The method of claim 8 wherein said suspected cancer cells are brain cancer cells and nervous system cancer cells.

12. The method of claim 8 wherein said suspected cancer cells are prostate cancer cells.

13. A method for preventing the onset of cancer and for preventing the recurrence of cancer comprising the administration of a therapeutic dose to a human recipient of one or more compositions selected from the group consisting of:
2-methoxyestradiol;
the 2-ethyl-17-β-estradiol molecules identified as analogues 20-22 in FIG. 3, specifically excluding any claim to 2-methyloxyestradiol;
the 17-α-ethynyl molecules identified as analogues 23-26 in FIG. 3;
the 17-α-ethyl molecules identified as analogues 27-30 in FIG. 3;
the 2,3-methylenedioxy molecules identified as analogues 31, 32, and 33 in FIG. 4;
the 2-alkoxy substituted analogues of estrone molecules identified as analogues 8-10 in FIG. 1;
the 2-ethyl substituted molecule identified as analogue 14 in FIG. 1; and
the 2,3-methylenedioxyestrone molecule identified as analogue 18 in FIG. 2.

14. The method of claim 13 wherein a therapuetic dose of eugenol is administered in conjunction with said one or more compositions.

15. The method of claim 13 wherein said cancer is human prostate cancer.

16. The method of claim 14 wherein said cancer is human prostate cancer.
